(19) United States
(12) Patent Application Publication
(10) Pub. No.: US 2026/0240447 A1
(43) Pub. Date: Aug. 20, 2026

(54) MULTI-MODALITY CONDITIONED VARIATIONAL U-NET FOR FIELD-OF-VIEW EXTENSION IN BRAIN DIFFUSION MRI

(71) Applicant: Vanderbilt University, Nashville, TN (US)

(72) Inventors: Zhiyuan Li, Nashville, TN (US); Tianyuan Yao, Nashville, TN (US); Praitayini Kanakaraj, Nashville, TN (US); Bennett A. Landman, Nashville, TN (US)

(21) Appl. No.: 19/540,378

(22) Filed: Feb. 13, 2026

Related U.S. Application Data
(60) Provisional application No. 63/758,784, filed on Feb. 14, 2025.

(51) Int. Cl. A61B 5/055

(52) U.S. Cl. CPC A61B 5/055; G06T 2207/20084

(57) ABSTRACT

A system for extending the field of view (FOV) of diffusion-weighted imaging (DWI) images of a patient having an incomplete FOV. A content module (e.g., a VAE encoder) is trained to extract diffusion features from each DWI slice, a shape module (e.g., a U-Net encoder) is trained to extract shape information from the extracted diffusion features and multi-modal data of the patient (e.g., a T1w image having a complete FOV, a DTI orientation map, etc.), and a spatial broadcast decoder (e.g., a U-Net decoder) is trained to generate a synthesized DWI image slice having a complete FOV based on the encoded features extracted from each DWI image slice and the multi-modal data. By explicitly disentangling “content” from “shape”, the dual-path architecture ensures that distinct information is utilized from different modalities. Meanwhile, the system effectively applies global diffusion features by broadcasting them to each pixel of the synthesized DWI image slice.

### CROSS-REFERENCE TO RELATED APPLICATIONS

This application claims priority to U.S. Prov. Pat. Appl. No. 63/758,784, filed Feb. 14, 2025, which is hereby incorporated by reference.

### FEDERAL FUNDING

This invention was made with government support under project numbers EB017230, EB032898, and AG074855 awarded by the U.S. National Institutes of Health and under grant number 1452485 awarded by the U.S. National Science Foundation. The government has certain rights in the invention.

### BACKGROUND

Diffusion magnetic resonance imaging (dMRI) provides an in-vivo and non-invasive approach that measures the displacement of water molecules in biological tissues and has become an essential technique for studying the microstructure and connectivity of brain white matter. Typically, dMRI scans need to acquire multiple volumes for capturing water diffusivity in different directions and thereby producing diffusion-weighted images (DWI). For each volume, a specific direction of diffusion-encoding magnetic gradient pulses will be applied to reflect the tissue properties that restrict the movement of water molecules. The larger diffusivity of water molecules along the gradient direction, the larger signal attenuation will be observed. This gradient direction is commonly described by a unit-length vector known as the b-vector. The amount of the attenuation is characterized by a variable called a b-value, which depends on the time and strength of the gradient pulse. Reference volumes, where b-value is set to 0 and therefore show no signal attenuation, are labeled as b0 images and are required for dMRI scans to provide a baseline signal intensity. Various metrics, like mean diffusivity (MD) and fractional anisotropy (FA), are calculated from these multi-volume DWIs using a diffusion tensor model to quantify water diffusion in brain tissues. Additionally, downstream analysis like white matter tractography can be conducted to offer insights into the whole-brain connections. Over the past ten years, dMRI and its associated analysis have become preferred approaches for investigating brain tissue characteristics for Alzheimer's diseases, stroke, schizophrenia, and the effects of aging.

One major challenge of dMRI in practice is the long acquisition time needed to capture a number of volumes at various gradient directions. dMRI protocols typically suggest using more than 31 directions for detailed studies on disease progression or treatment effects. That extended acquisition time can further introduce and amplify imaging artifacts, such as patient motion and eddy-current induced distortions, and consequently resulting in dMRI scans that have incomplete FOV, which is one of the most prevalent problems detected during dMRI data quality checks.

In a recent analysis of dMRI datasets, 103 out of 1057 cases exhibited incomplete FOV due to such challenges. Gao et al., “Field-of-view extension for diffusion MRI via deep generative models,” Journal of Medical Imaging, Vol. 11, Issue 4, 044008 (August 2024), https://doi.org/10.1117/1.JMI.11.4.044008. FIG. 1A is a visualization and FIG. 1B is a histogram of the 103 real cases of dMRI scans with incomplete FOV that failed quality assurance. In FIG. 1A, horizontal regions indicate the distribution of the incomplete part of FOV with an estimated position of a brain mask. The total cutoff distance from the incomplete FOV to the top of the brain is estimated using a corresponding and registered T1-weighted (T1w) image.

The absence of data from these missing regions not only hinders analyses in the incomplete part of the FOV but also impacts the accuracy of tractography across the entire brain. As shown in FIGS. 1C-1E, an incomplete FOV not only makes it impossible to analyze the missing regions (e.g., as illustrated in FIG. 1C), but can also impact the tractography performed in the acquired regions, for example resulting in missing streamlines of various tract (CST) compared with reference (e.g., as illustrated in FIG. 1E). Although existing works can somewhat impute the missing regions, they encounter difficulty when imputing slices close to the top edge of the brain, which therefore affects the tractography in those regions (e.g., as illustrated in FIG. 1D). That corrupted data then leave gaps among the complete analysis of patients and introduces significant obstacles in diagnosing and monitoring neurological conditions like Alzheimer's Disease.

Traditional imputation methods for dMRI fail to address the FOV extension tasks due to the requirement of multi-volumes reference signals, which are however unavailable in the incomplete part of the FOV across all volumes. Deep learning methods have shown promising performance for medical image synthesis tasks such as distortion correction, denoising, and registration. Meanwhile, information from other modalities has proven to be beneficial in enhancing the quality of synthesized images. For example, T1w images are widely used for providing anatomical reference for many dMRI synthesis tasks.

Aligning with these approaches, Gao et al. demonstrated that a basic image-to-image translation artificial neural network conditioned on T1w images effectively enables the imputation of a large range of incomplete FOV in dMRI, That imputation is then useful for improving the accuracy of whole brain tractography. That straightforward approach, however, exhibits three primary limitations.

First, that baseline method struggles to impute slices near the top edges of the brain (e.g., as shown in FIG. 1E). Second, that baseline method treats the information from T1w images and dMRI images equally by simply concatenating them in the input layer of the neural network, raising questions about the optimal use of the unique information from different modalities and how to specifically utilize different information for improving the quality of dMRI imputations. Third, other low-hanging conditions for dMRI (like b-vector and diffusion tensor images (DTI)) are not considered as a group of multi-modality conditions besides T1w images. In summary, because the diffusion features in a single DWI volume are highly consistent under the same b-value and b-vector, the diffusion features observed in the acquired regions should be critical for accurately imputing the incomplete part of the FOV in the same volume. Therefore, there is a need for more advanced methods of extending the field of view of acquired dMRI images that take advantage of the available anatomical structure information from other available modalities.

### SUMMARY

The disclosed system includes a number of novel and nonobvious features that, particularly in combination, overcome those and other technical drawbacks of the baseline method.

Rather than treating all input modalities equally (relying on a network to implicitly figure out which input controls shape and which controls texture), the disclosed system uses a novel dual-path architecture that explicitly disentangles “content” from “shape”. A content module (e.g., VAE encoder) extracts diffusion features (e.g., a global z-vector) while a separate shape module (e.g., U-Net encoder) processes the anatomical data. That dual-path architecture ensures that the system specifically utilizes distinct information from different modalities, rather than blurring them together.

Additionally, rather than conditioning the imputation only on T1w images, embodiments of the disclosed system integrates DTI orientation maps and b-vector maps into the shape module, providing a calculated prior of the fiber direction (not just the tissue density) and allowing the disclosed system to accurately reconstruct darker voxels in white matter (representing signal attenuation along fiber tracts) that the baseline method fails to capture.

Additionally, rather than processing information locally using a standard convolutional U-Net, which struggle to consistently apply global constraints (for instance, that an entire volume of sagittal slices has a constant b-value) across an entire image, embodiments of the disclosed system tile the global z-vector to form a z-map, explicitly broadcasting the global z-vector to every single pixel coordinate in the target grid and introducing a structured prior that forces the U-Net to globally and uniformly apply the learned diffusion features to the anatomical structure.

For at least those reasons, the disclosed system more effectively fills in the brain shape represented in the multi-modal data with the diffusion content extracted from the acquired DWI image slices. Meanwhile, by improving the completeness and accuracy of whole brain tractography, the disclosed system provides a more reliable technique for potential clinical studies and interventions.

### BRIEF DESCRIPTION OF THE DRAWINGS

Aspects of exemplary embodiments may be better understood with reference to the accompanying drawings. The components in the drawings are not necessarily to scale; emphasis instead being placed upon illustrating the principles of exemplary embodiments.

FIG. 1A is a visualization of diffusion magnetic resonance imaging (dMRI) scans with incomplete fields of view (FOV) that failed quality assurance.

FIG. 1B is a histogram illustrating the incomplete FOV of the dMRI scans visualized in FIG. 1A.

FIG. 1C illustrates the acquired regions and missing regions of an example dMRI scan with an incomplete FOV.

FIG. 1D illustrates the imputed regions of the example dMRI scan of FIG. 1C using a baseline method.

FIG. 1E illustrates a reference image showing the acquired regions missing from the example dMRI scan of FIG. 1C and imputed using the baseline method of FIG. 1D.

FIG. 2 is a block diagram illustrating a system according to exemplary embodiments.

FIG. 3 is a diagram illustrating the content module of FIG. 2 according to exemplary embodiments.

FIG. 4 is a diagram illustrating the shape module and spatial broadcast decoder of FIG. 2 according to exemplary embodiments.

FIG. 5 illustrates an axial view of the imputations for b1300 images

FIG. 6 is a visualization of tracts computed from images imputed by a baseline method and the disclosed system.

FIG. 7 is a comparison of Dice scores for the baseline method and the disclosed system.

FIG. 8A are Bland-Altman plots for examining the shape measurements of Alzheimer's Disease-associated bundles.

FIG. 8B are additional Bland-Altman plots for examining the shape measurements of Alzheimer's Disease-associated bundles.

FIG. 8C are additional Bland-Altman plots for examining the shape measurements of Alzheimer's Disease-associated bundles.

FIG. 8D are additional Bland-Altman plots for examining the shape measurements of Alzheimer's Disease-associated bundles.

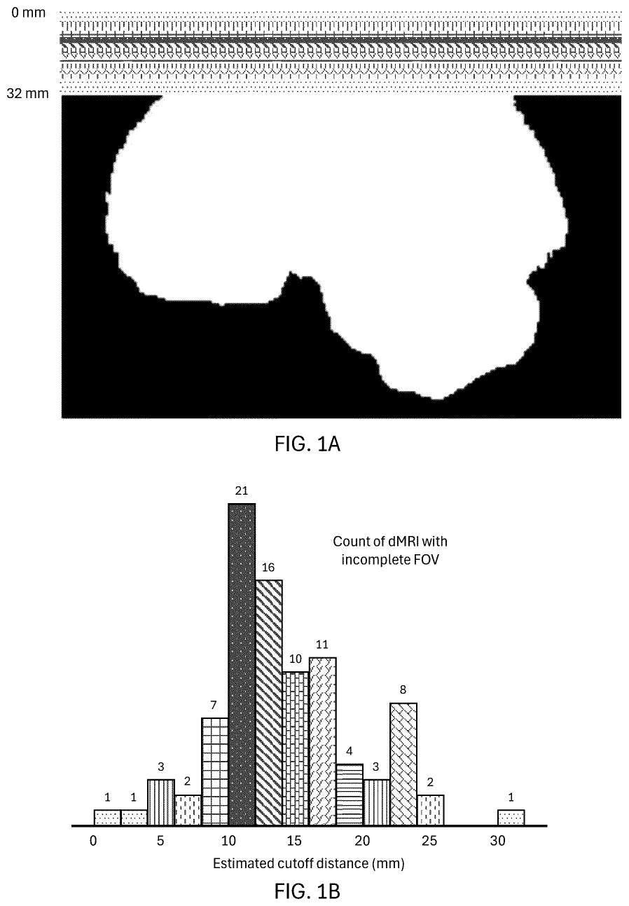

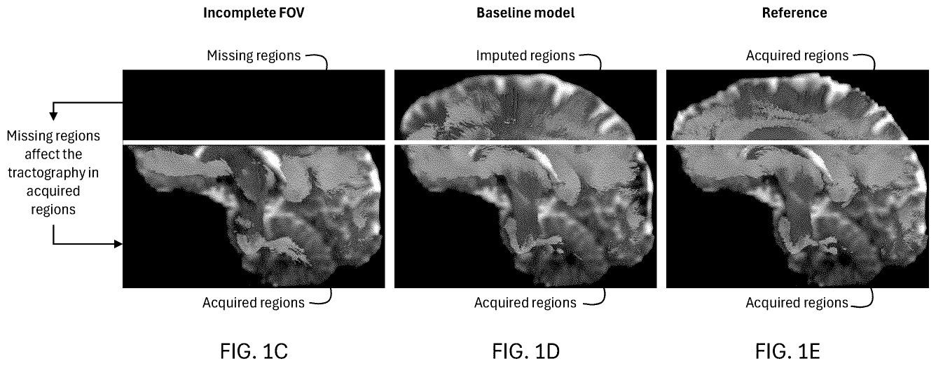

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

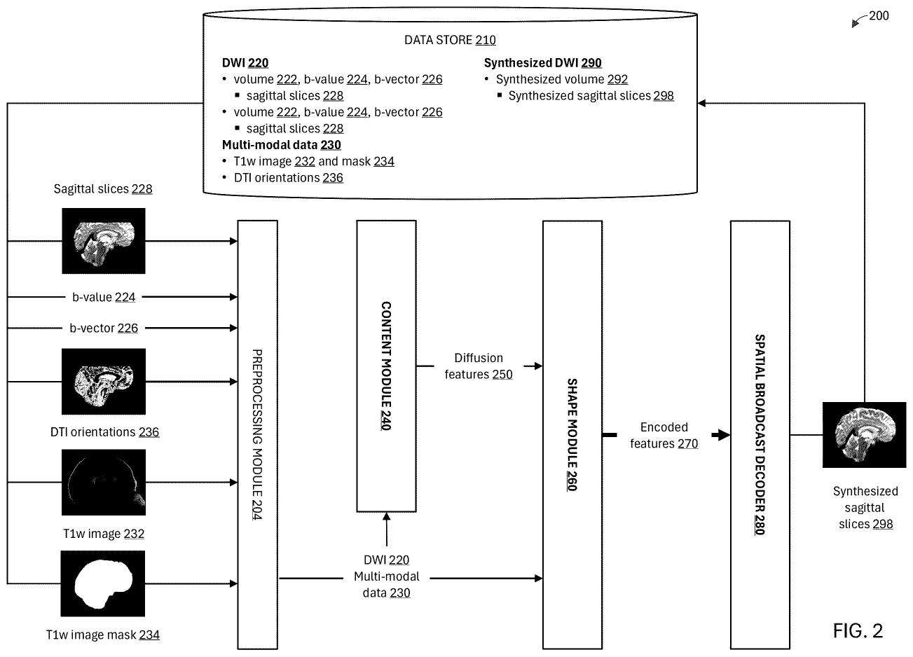

FIG. 2 is a block diagram illustrating a system 200 for extending the field of view (FOV) in brain diffusion magnetic resonance imaging (dMRI) scans.

In the embodiment of FIG. 2, the system 200 includes a data store 210 (realized as non-transitory computer readable storage media), a pre-processing module 204, a content module 240, a shape module 260, and a spatial broadcast decoder 280.

The data store 210 stores diffusion-weighted images (DWI) 220 with an incomplete FOV and multi-modal data 230. As briefly outlined above, each diffusion-weighted image 220 includes multiple volumes 222, each captured with a specific b-value 224 (indicative of the observed signal attenuation caused by diffusion-encoding magnetic gradient pulses) and, when the b-value 224 is non-zero, a b-vector 236 indicative of the direction of the diffusion-encoding magnetic gradient pulses. Each volume 222 is partitioned into slices. In the embodiment of FIG. 2, each volume 222 is partitioned into sagittal slices 228.

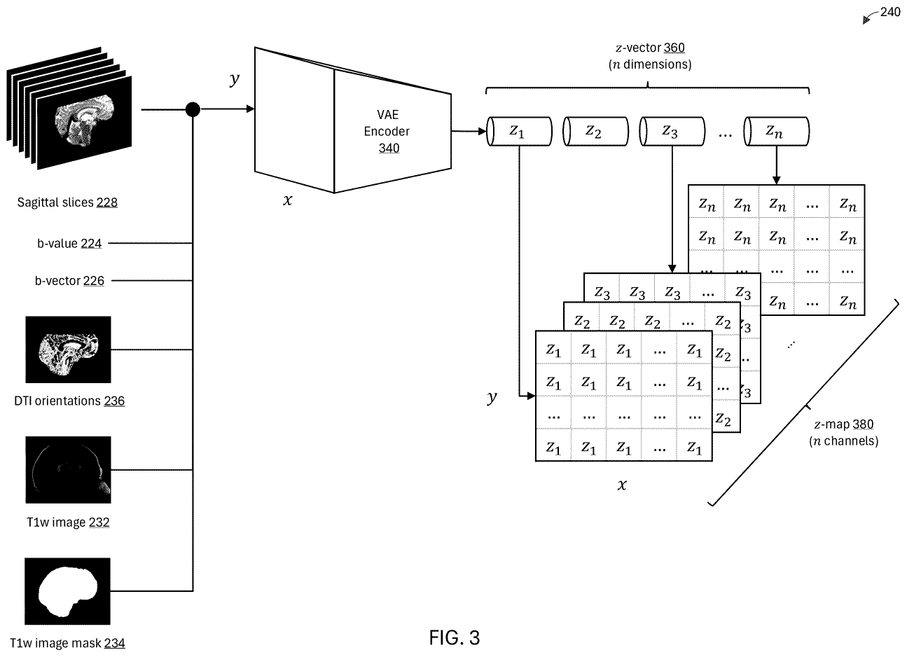

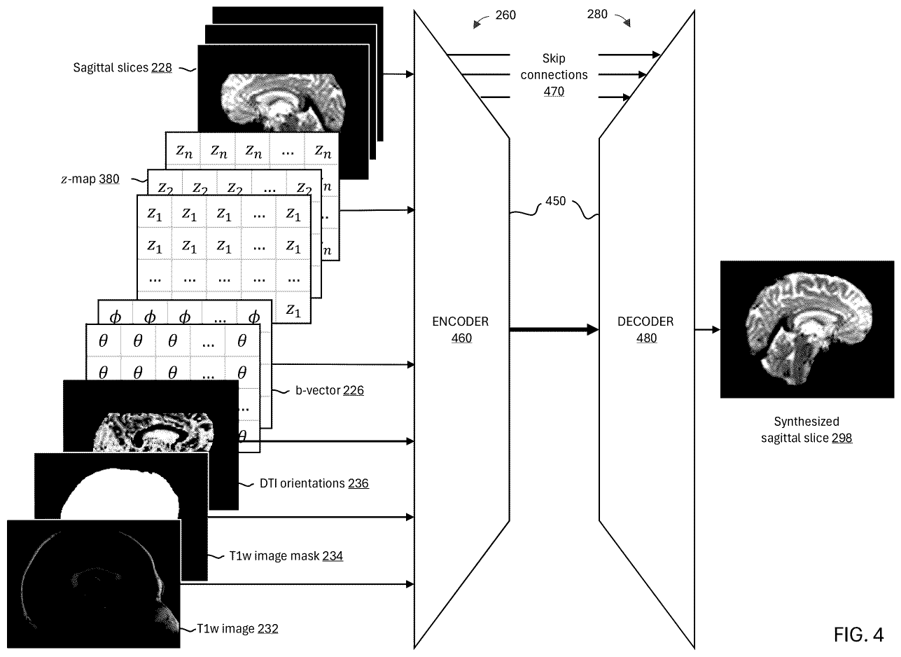

As described in more detail below, the system 200 extends the field of view of each sagittal slice 228 by generating a synthesized sagittal slice 298 having a complete field of view. To generate each synthesized sagittal slice 298, the corresponding sagittal slice 228 and multi-modal data 230 are provided to a content module 240, which extracts diffusion features 250 as described in detail below with reference to FIG. 3. Those extracted diffusion features 250 and the multi-modal data 230 are then provided to a shape module 260 that extracts shape information and outputs encoded features 270 to a spatial broadcast decoder 280 that, as described in detail below with reference to FIG. 4, generates the synthesized sagittal slice 298 having a complete field of view. Each set of synthesized sagittal slices 298 from each DWI volume 222 are then stored in the data store 210 to form a synthesized volume 292 having a complete field of view. Each synthesized volume 292 generated from each DWI 220 is then stored in the data store 210 to form a synthesized DWI having a complete field of view.

In the embodiments of FIG. 2, the multi-modal data 230 includes a T1-weighted (T1w) image 232 having a complete field of view, an image mask of the T1w image 232 (T1w image mask 234), and a diffusion tensor image (DTI) orientation map 236. A T1-weighted (T1w) image 232 is a standard structural MRI scan that provides a high-resolution, three-dimensional map of the brain anatomy of the patient. Even in scenarios where the diffusion-weighted images 220 of a patient have an incomplete field of view (e.g., for the reasons described above), that same patient often has a T1w image 232 with a complete field of view. Providing the T1w image 232 to the shape module 260 enables the shape module 260 to extract shape features indicate of the location of brain structures (like the skull, white matter, and gray matter) in the missing region. A T1w image mask 234 is a binary image derived from the T1-weighted scan that delineates the boundaries of the brain. Providing the T1w image mask 234, which delineates the boundaries of the brain, ensures that the system 200 only processes and evaluates valid brain tissue while ignoring background elements.

A DTI orientation map 236 is a computed map visualizing the primary direction of water diffusion in the brain tissues. In fibrous tissues like white matter, water diffuses faster along the fiber than across it. DTI orientations indicate the angle of that fiber tract at each specific pixel. DTI orientation maps 236 are typically represented as a color-coded image, where the color indicates the axis of diffusion. For example, red may be indicative of the left-right orientation, green may be indicative of an anterior-posterior orientation, and blue may be indicative of superior-inferior orientation. The DTI orientations may be derived from multiple volumes 222 of the acquired DWI 220 (having multiple b-values 224 and b-vectors 226). In those embodiments, the DTI orientations may be derived by fitting a diffusion tensor model (e.g., an ellipsoid) to each voxel to determine which shape best fits the water movement, calculating the major eigenvector (e.g., the longest axis of the ellipsoid), and storing the angles of that major eigenvector as the three channels (e.g., red, green, and blue) of the DTI orientation map 236. By providing the fiber direction in the acquired regions of the DWI 220, the DTI orientation map 236 enables the system 200 to better infer how those fibers should continue into the missing regions.

In embodiments, the preprocessing module 204 may process the DWI 220 by performing denoising, solving inter-volume motion, fixing dropout slices, and correcting artifacts induced by susceptibility and eddy current. The preprocessing module 204 may also separately normalize the intensity of the DWI 220 and the T1w image 232. For example, the maximum intensity value may be adjusted to the 99.9th percentile, and the minimum value may be set to zero. The DWI 220 may be resampled to isotropic 1 mm space, for example using the MRtrix3 “mrgrid” command. The T1w image 232 may be registered to that isotropic 1 mm space DWI 220, for example using FSL's epi reg (that applies an affine transformation computed between the T1w image 232 and the average b0 image of the DWI 220). The DTI orientations of the acquired regions may be computed for each DWI 220. Accordingly, the DTI orientation map 236 may have the same incomplete FOV as the DWI 220. Finally, a b-vector 226 map may be created for each volume 222. The b-vector 226 map, for example, may be a two-channels image formed by tiling the polar coordinates (θ and φ) of the b-vector 226. Some multi-modality data 230, such as T1w image 232, may be used for all volumes 222 of the DWI 230. Other multi-modality data 230, such as the b-vector 226 maps, are specific to each volume 222.

By partitioning each volume 222 into slices that are substantially orthogonal to missing slices, the disclosed system 200 is able to impute the missing regions and generate a synthesized volume 282 having a complete field of view. In the example of FIG. 2, for instance, some axial slices of the volume 222 were not acquired. By partitioning the volume 222 into sagittal slices 228 (or coronal slices) that are substantially orthogonal to those missing axial slices, the claimed system is able to form synthesized coronal or sagittal slices 288 (and a synthesized volume 282) having a complete field of view by using the acquired regions of each coronal or sagittal slice 228 to impute the missing regions in each coronal or sagittal slice 228.

FIG. 3 is a diagram illustrating the content module 240 for extracting diffusion features 250 according to exemplary embodiments.

In the embodiment of FIG. 3, the content module 240 is realized as a variational autoencoder (VAE) encoder 340. As shown in FIG. 3, the sagittal slice 228 (having dimensions x×y) and the multi-modal data 230 are provided to the VAE encoder 340, which generates an n-dimensional z-vector 360. In some embodiments, the sagittal slice 228 is provided along with the surrounding sagittal slices (e.g., the 5 neighboring slices in each direction) to provide context. Each dimension zᵢ of the extracted z-vector 360 is tiled to form an array having the same dimensions (x×y) as the sagittal slice 228. The n arrays form a z-map 380 having n channels.

The VAE encoder 340 is a neural network inference model that maps input data to a probabilistic latent space rather than a fixed vector. Specifically, instead of outputting a single code, the VAE encoder 340 outputs the parameters (typically mean μ and variance σ²) of a probability distribution (usually a Gaussian). The latent feature vector (the z-vector 360) is then sampled from that distribution. The VAE encoder 340 architecture allows the model to learn a continuous, structured latent space where the distribution of features approximates a prior (typically an isotropic Gaussian), optimized by minimizing the Kullback-Leibler (KL) divergence between the inferred posterior and the prior.

In the disclosed system 200, the diffusion features 250 (represented as the latent z-vector 360) characterize the specific signal intensity and texture properties of the acquired DWI slices 228. Those diffusion features 250 represent the “content” of the input sagittal slice 228 (specifically, the signal attenuation caused by water diffusion at the specific b-value 224 and gradient direction of that volume 222). Those diffusion features 250 are distinct from the “shape” or anatomical structure of the brain.

The diffusion features 250 are encoded into a single n-dimensional latent z-vector 360, which provides a global representation of the diffusion characteristics of the input sagittal slice 228. For the extraction process, the VAE encoder 340 may utilize a Conditional VAE (CVAE) architecture to isolate diffusion features 250 from anatomical information. To prepare the input x¿ for each target sagittal slice 228, the system 200 may construct a “2.5D” volume consisting of the target sagittal slice 228 concatenated with its 5 neighboring slices on either side (totaling 11 slices) to capture volumetric context

The system 200 pairs that DWI 220 input x with the corresponding images Y from the multi-modality data 230 (e.g., the T1w image 232, the T1w image mask 234, the DTI orientation map 236, the b-vector map 226). The VAE encoder 340, which is parameterized by @, receives the concatenated pair of the acquired DWI x and the multi-modal condition Y. The VAE encoder 340 is trained on reference data (e.g., DWI with regions removed) to learn an inference model q^{φ}(z|x, Y) for extracting each z-vector 360. By using the multi-modal data 230 to condition the VAE encoder 340 on the anatomy of the patient, the system 200 trains the VAE encoder 340 to extract the residual variation (e.g., the diffusion-specific intensity features) that is not explained by the static anatomy.

The VAE encoder 340 outputs the distribution parameters and the system 200 samples the latent feature vector z from that distribution. The z-vector 360 is the final set of extracted diffusion features 250 used for the broadcasting and decoding steps described below.

FIG. 4 is a diagram illustrating the shape module 260 for extracting shape features and the spatial broadcast decoder 280 for generating synthesized sagittal slices 298 according to exemplary embodiments.

In the embodiment of FIG. 4, the shape module 260 is realized as a U-Net encoder 460 and the spatial broadcast decoder 280 is realized as a U-Net decoder 480 having skip connections 470 from the U-Net encoder 460. The U-Net encoder 460 and decoder 480 are collectively referred to herein as U-Net 450.

The U-Net 450 is a convolutional neural network (CNN) architecture designed for biomedical image segmentation. The encoder-decoder structure with skip connections 470 enables precise pixel-level localization by capturing context and maintaining high-resolution features. As illustrated in FIG. 4, the U-Net encoder 460 captures context through convolution and down-sampling (max-pooling) layers and outputs feature maps to the U-Net decoder 480, which up-samples those feature maps to enable precise localization. The skip connections 470 directly connect encoder layers in the U-Net encoder 460 to decoder layers in the U-Net decoder 480, ensuring that high-resolution, high-frequency details from the input that are crucial for accurate segmentation (like edges) are preserved in the output.

To synthesize each sagittal slice 298, the z-map 380 is concatenated with the sagittal slices 228 (x) and the corresponding multi-modality data Y (e.g., the T1w image 232, the T1w image mask 234, the DTI orientations 238, and the b-vector 226 map for the corresponding volume 222 tiled in an array having the same dimensions as the sagittal slice 228). Accordingly, the diffusion features 250 extracted from the acquired sagittal slices 228 are spatially aligned with the anatomical structure introduced by the multi-modality data 230. Meanwhile, by tiling the global diffusion features (the z-map 380) across the spatial dimensions of the shape features, the system 200 effectively “broadcasts” the specific signal attenuation style of the volume to every pixel of the anatomical structure.

The U-Net 450 is parameterized by θ. Similar to the VAE encoder 340, the U-Net 450 is trained on reference data (e.g., DWI with regions removed) to learn a generative model p(x|z, Y) for synthesizing sagittal slices 298 that addresses any imputation issues in the incomplete parts of the FOV. The VAE encoder 340 parameter φ and the U-Net 450 parameter θ may be optimized by the evidence lower bound (ELBO) as:

$${ℒ}_{VAE} ( θ , ϕ ) = {𝔼}_{{q}_{ϕ} ( z \vert x , Y )} [ log {p}_{θ} ( x \vert z , Y ) ] - {D}_{KL} [ {q}_{ϕ} ( z \vert x , Y ) \vert \vert p ( z \vert Y ) ] \qquad [ Equation 1 ]$$

where the first term is the expectation of log-likelihood of the observed DWI with complete FOV, which is implemented as reconstruction loss of the imputation regions. The second term is KL divergence between the inferred posterior distribution of the diffusion features and its prior distribution, which is implemented as an isotropic Gaussian distribution parameterized as N(0,I).

To enhance the realism of the generated images, some embodiments additionally utilize the generative adversarial network (GAN) objective as follows:

$${ℒ}_{GAN} ( θ , D ) = {𝔼}_{x} [ log D ( x ) ] + {𝔼}_{x} [ log ( 1 - D ( {p}_{θ} ( x \vert z , Y ) ) ] \qquad [ Equation 2 ]$$

where D is a discriminator to criticize whether the output of generative model G looks real. In those embodiments, the final objective of the disclosed model is formulated as:

$$ℒ = {ℒ}_{GAN} ( θ , D ) + {ℒ}_{VAE} ( θ , ϕ ) \qquad [ Equation 3 ]$$

In either embodiment, the loss (defined in Equation 1 or Equation 3) is then computed between the synthesized sagittal slices 288 generated by the system 200 and the ground truth images in the reference data.

### Benefits of the Disclosed Invention

As described above, the baseline method described in Gao, et al. treats all input modalities equally (i.e., taking the anatomical T1w image and the diffusion image and simply concatenates them together at the input layer of a standard neural network). Accordingly, the Gao method relies on the network to implicitly figure out which input controls shape and which controls texture. By contrast, the disclosed system 200 uses a novel dual-path architecture that explicitly disentangles “content” from “shape”. The content module 240 (e.g., VAE encoder 340) extracts diffusion features 250 (e.g., a global z-vector 360) while a separate shape module 260 (e.g., U-Net encoder 460) processes the anatomical data. That dual-path architecture ensures that the system 200 specifically utilizes distinct information from different modalities, rather than blurring them together.

Additionally, by conditioning the imputation only on T1w images, the baseline method of Gao et al. ignores all other available geometric data. By contrast, the disclosed system 200 integrates DTI orientation maps 236 and b-vector maps 226 into the shape module 260. Utilizing the DTI orientation maps 236 provides a calculated prior of the fiber direction (not just the tissue density), allowing the system 200 to accurately reconstruct darker voxels in white matter (representing signal attenuation along fiber tracts) that the baseline method fails to capture.

Additionally, standard U-Nets (e.g., as used in the baseline method of Gao, et al.) are convolutional, meaning they process information locally. Accordingly, standard U-Nets struggle to consistently apply global constraints (for instance, that an entire volume of sagittal slices 228 has a constant b-value 224) across an entire image. That deficiency leads to poor performance near the edges of the brain where local context is missing (known as the “top edge” problem). By tiling the global z-vector 360 to form the z-map 380, the disclosed system 200 overcomes that drawback explicitly broadcasting the global z-vector 360 to every single pixel coordinate in the target grid. Broadcasting that global z-vector 360 introduces a structured prior, forcing the U-Net to globally and uniformly apply the learned diffusion features 250 to the anatomical structure. Therefore, the disclosed system 200 more effectively fills in the brain shape represented in the multi-modal data 230 with the diffusion content extracted from the acquired sagittal slices 228. Meanwhile, by improving the completeness and accuracy of whole brain tractography, the disclosed system 200 provides a more reliable technique for potential clinical studies and interventions.

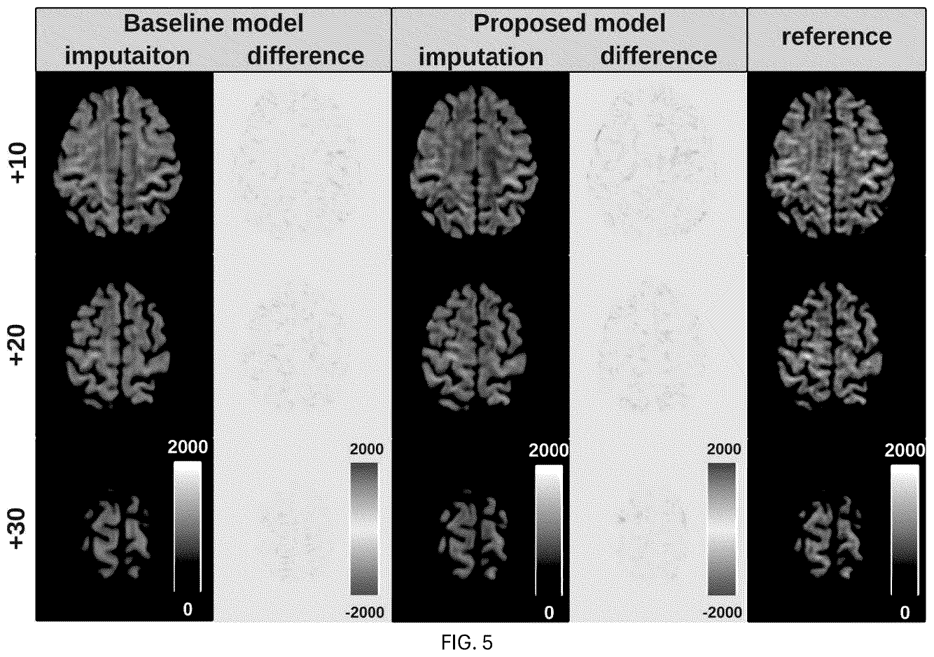

For at least those reasons, the novel design of the disclosed system 200 achieves a more accurate volumetric imputation that better reflects the signal attenuation in dMRI due to the white matter tissue properties, which is supported by both the qualitative visualization (FIG. 5) and quantitative volumetric metric, ACC of the fODF (Table 1) described in the experimental results below. Those results confirm the hypothesis that by explicitly integrating distinct information from multi-modality images, the disclosed system 200 and method can improve the imputation of the incomplete FOV in dMRI.

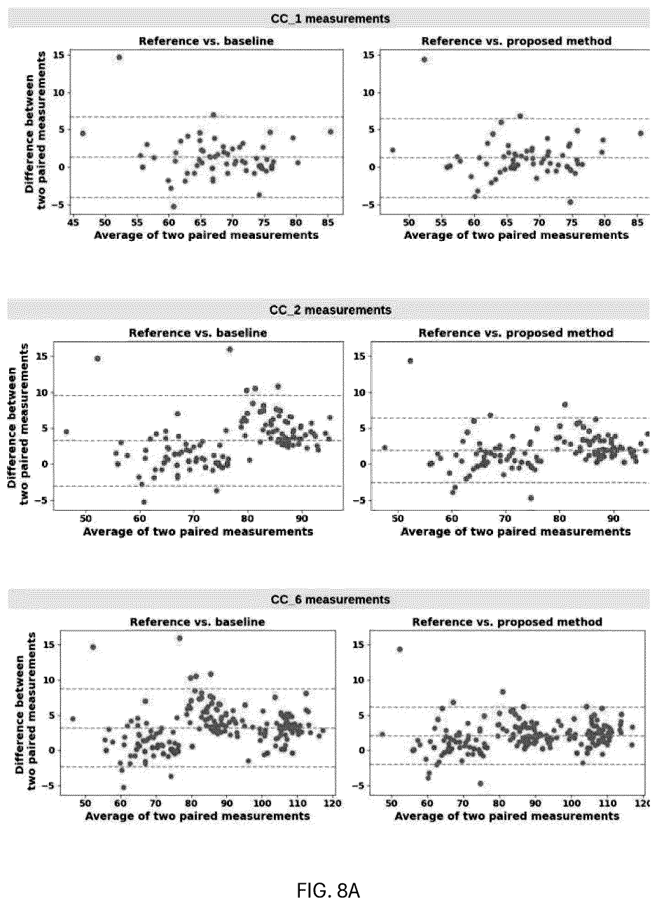

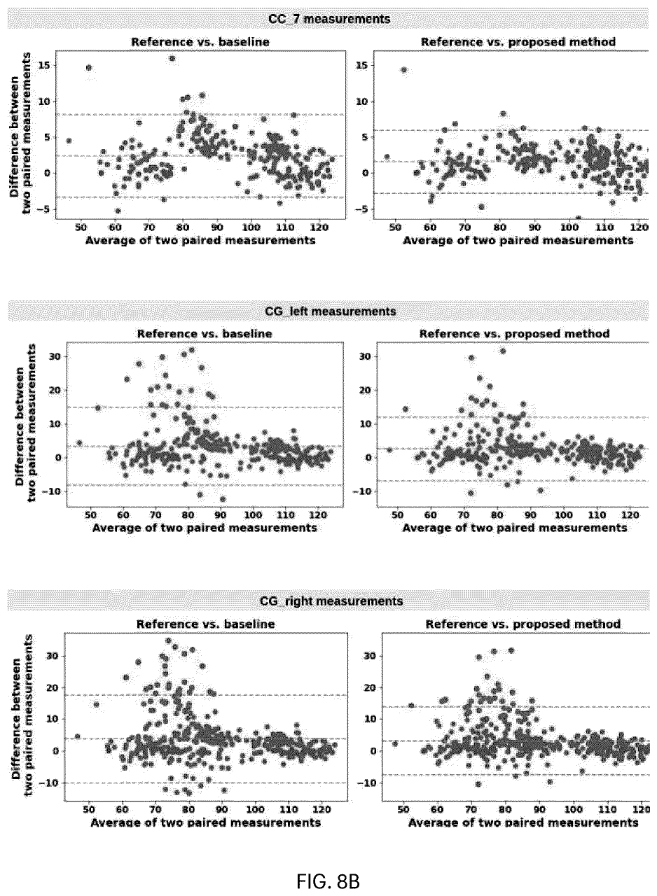

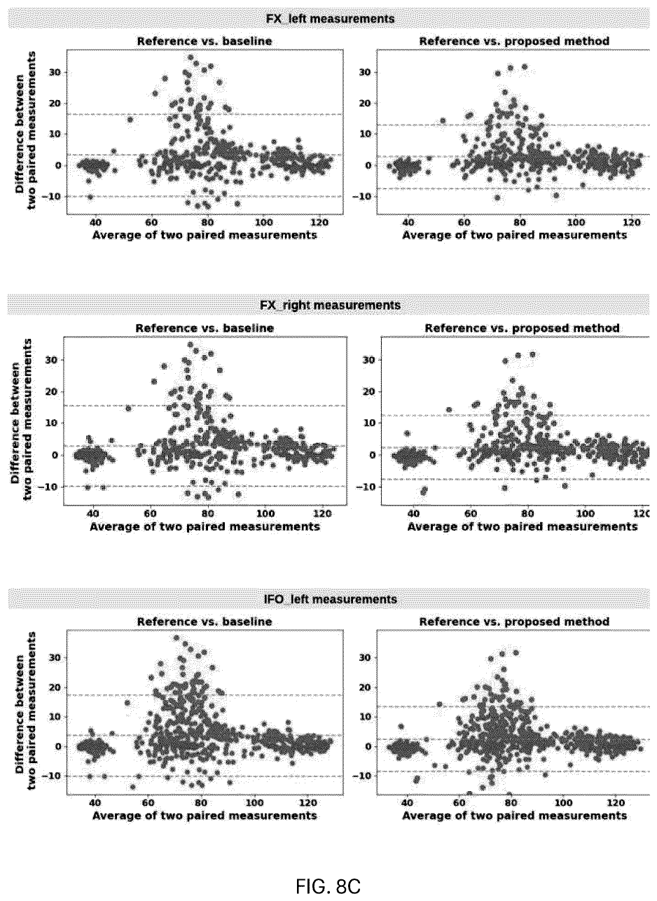

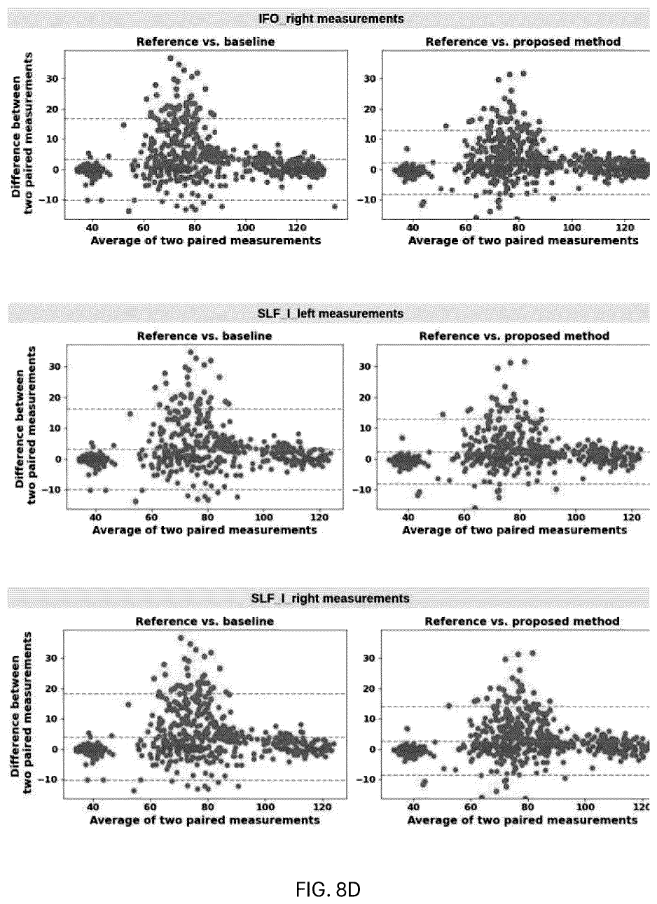

Finally, for neurodegenerative disorders like Alzheimer's Disease, where early detection and accurate monitoring of disease progression are crucial, the ability to accurately reconstruct and analyze brain tracts can significantly influence therapeutic strategies and outcomes, especially when dealing with dMRI data that may contain incomplete FOV. By providing a more reliable imputation technique, the disclosed system 200 is useful for repairing incomplete data and further aids in a deeper understanding of AD pathology and its impact on brain connectivity. Specifically, in the experimental results described below, the reliability of the disclosed approach is demonstrated through Bland-Altman plots for tracts associated with AD (FIGS. 8A-8D), which revealed a more consistent agreement with reference tract measurements, highlighting the potential of the disclosed approach to reduce diagnostic uncertainties and enhance monitoring of disease progression by fixing valuable dMRI data.

### EXPERIMENTAL RESULTS

The following studies were conducted to evaluate the imputation performance of the disclosed method and its usefulness on improving the accuracy of whole brain white matter bundles. To test the hypothesis that the disclosed method can address the limitations of existing works for imputing dMRI by specifically integrating distinct information from multi-modality images, Gao et al. was chosen as the baseline.

Similar to Gao et al., the Wisconsin Registry for Alzheimer's Prevention (WRAP) dataset was chosen as the primary resource for training and evaluating the disclosed methodologies for two main reasons. First, the data from WRAP was collected at a single site, providing a consistent setting for training and evaluating models without the complications of inter-site variability. Second, the WRAP dataset includes some of the most extensively corrupted dMRI data, with missing brain regions nearly 30 mm in length due to an incomplete FOV. The initial cohort for the WRAP study included 323 participants, all of whom had T1w image and single-shell dMRI scans with a b-value of 1300 s/mm², which is the most common b-value acquired in WRAP protocols. Those participants were divided into three groups:231 in the training set, 46 in the validation set, and 46 in the testing set. To further assess the robustness and generalizability of the disclosed method, the study was expanded to incorporate the National Alzheimer's Coordinating Center (NACC) dataset, which contains a substantial number of dMRI scans also acquired at a b-value of 1300 s/mm². The second cohort included 50 test subjects from NACC, each with T1w image and single-shell dMRI scans at b-value of 1300 s/mm².

First, to quantitatively evaluate the error of the imputation, SLANT-TICV was used to compute a brain mask and apply it to the imputed brain to ensure that only brain areas are considered for computing metrics. Quantitatively, for the imputed brain regions, the voxel-wise peak signal-to-noise ratio (PSNR) and the angular correlation coefficients (ACC) of the white matter fiber orientation distribution function (fODF) estimated from all DWI volumes is reported. Additionally, visualization of the imputed slices for qualitative evaluation is presented.

Next, to test the hypothesis that an improved imputation of the incomplete part of FOV can improve the whole brain tractography, whole brain bundle analysis is conducted and the tracts produced from images imputed by the disclosed methods and the baseline methods are evaluated. To ensure an accurate comparison, tracts were extracted using Tractseg from images imputed by both the baseline and disclosed methods. Those tracts were then compared against the same tract segmentation derived from the ground truth reference image with a complete FOV, and the Dice score was calculated for each imputation method. Paired t-tests were conducted for the 72 tracts and present the visualization of the streamlines produced from different imputation methods and mark the difference and improvement compared with ground truth reference streamlines. Specifically, a group of 12 tracts are investigated that are commonly associated with Alzheimer's disease (AD), which is Rostrum (CC_1), Genu (CC_2), Isthmus (CC_6) and Splenium (CC_7) of the Corpus Callosum (CC) as well as left and right Cingulum (CG), Fornix (FX), Inferior occipitofrontal fascicle (IFO), and Superior longitudinal fascicle I (SLF_I), for exploring potential clinical benefits of the disclosed framework. Bland-Altman plots are presented to analyze the agreement in the shape measurements of these bundles between the reference and various imputation methods.

### Imputation of the Incomplete Part of the FOV

FIG. 5 illustrates an axial view of the imputations for b1300 images. Each row represents a certain distance to the nearest acquired slice in millimeters (mm). Red and blue indicate that the imputed intensity is larger or smaller, respectively, than the ground truth reference. Compared with the baseline model, the proposed model can impute voxel with smaller values (appears darker) for highly structured tissues, which better reflects the MRI signal attenuation due to the diffusion of water molecules.

TABLE 1
Average ACC of fODF and PSNR for the imputation regions of testing data on
WRAP and NACC datasets. The disclosed method achieved significant improvement
for all-volume-wise imputation, as demonstrated by the ACC values. No significant
difference is observed when imputing both white matter (WM) and non-WM regions,
as demonstrated by the voxel-wise based metric, PSNR.

| | | WRAP | | NACC | |
|---|---|---|---|---|---|
| ACC | baseline | 0.733 ± 0.039 | | 0.703 ± 0.075 | |
| | disclosed method | 0.798 ± 0.041 | | 0.731 ± 0.069 | |
| | p-value | * 1.8E−28 | | * 1.7E−6 | |
| | | b0 images | b1300 images | b0 images | b1300 images |
| PSNR | baseline | 34.30 ± 1.85 | 24.00 ± 1.52 | 32.77 ± 1.93 | 23.21 ± 1.34 |
| WM | disclosed method | 34.21 ± 1.93 | 24.45 ± 1.67 | 32.02 ± 2.32 | 22.81 ± 1.41 |
| | p-value | 0.029 | * 3.7E−96 | * 5.1E−26 | *5.6E−58 |
| PSNR | baseline | 28.91 ± 1.83 | 29.24 ± 1.37 | 27.66 ± 2.46 | 29.37 ± 1.26 |
| Non- | disclosed method | 28.10 ± 1.66 | 28.97 ± 1.42 | 27.31 ± 2.22 | 28.96 ± 1.27 |
| WM | p-value | * 2.1E−9 | * 0.0 | *3.1E−14 | * 0.0 |

The disclosed model, in comparison to the baseline model, can more accurately impute smaller voxel values (resulting in a darker appearance) for highly structured tissues in white matter. That improvement better represents the dMRI signal attenuation associated with water molecule diffusion along the directions of nerve fibers. That difference in imputation is clearly demonstrated by the volumetric metric, ACC of the fODF, where the disclosed method achieved relative improvements of 8.9% and 4.0% on the WRAP and NACC datasets, respectively; the improvement is statistically significant, as indicated by the small p-value (Table 1, top). Regarding the voxel-wise measurement, PSNR, the difference between the baseline method and the disclosed method is small in both white matter regions and non-white-matter regions (Table 1, bottom).

### White Matter Bundle Analysis

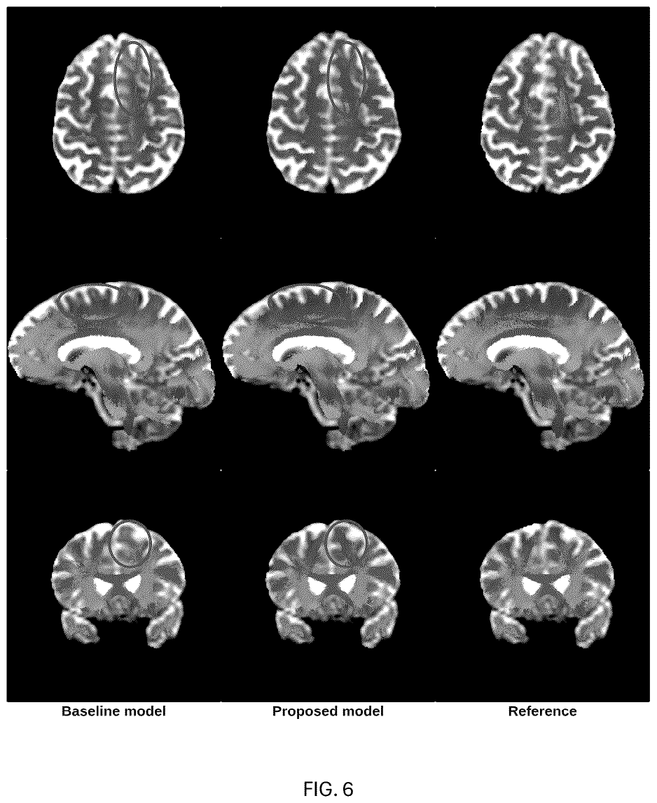

FIG. 6 is a visualization of tracts computed from images imputed by the baseline model and the proposed model. The proposed model allows for more accurate and complete tractography, as demonstrated by its ability to fix the broken and incomplete streamlines shown in the baseline model.

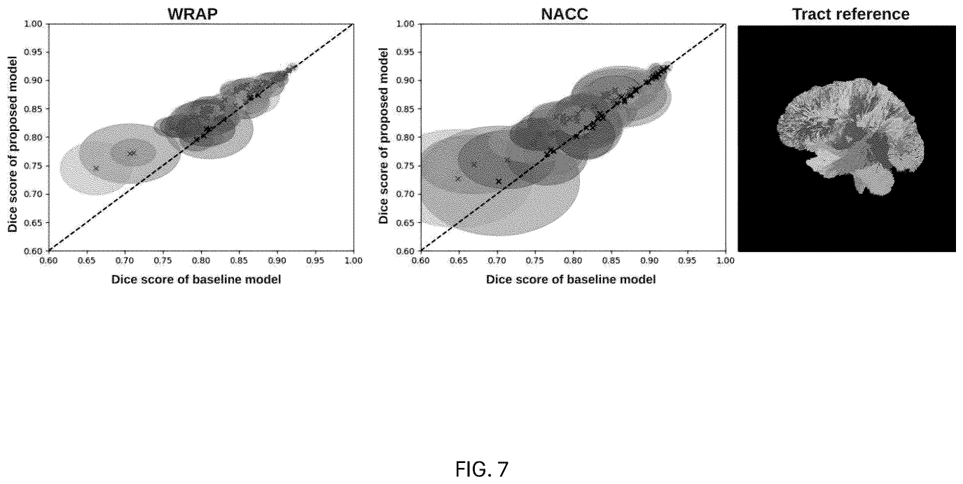

FIG. 7 is a comparison of Dice scores for the baseline model and the proposed model. In the left two figures, each colored ellipse represents one tract. The center of each ellipse is determined by the Dice scores of the baseline model (x-coordinate) and the proposed model (y-coordinate). The major and minor axes of the ellipse represent the standard deviation (STD) of the Dice scores for the baseline and proposed models, respectively. The corresponding reference for each tract is plotted in the right figure, where the colors of tracts match that in the left two figures. The proposed model consistently shows improvement for almost all tracts, as indicated by all ellipses are located above the black dashed line (y=x), which represents equal performance. For tracts where the difference is significant (p<0.01), the centers are colored red; otherwise, the centers are black.

FIGS. 8A-8D are Bland-Altman plots illustrating the agreement on average bundle length compared to the reference. Measurements within the best 10% for accuracy are marked in green, while those in the worst 10% are in red. The analysis specifically investigates the tracts associated with Alzheimer's Disease (AD), such as CC_1, CC_2, CC_6, CC_7, CG, FX, IFO, and SLF_I. The disclosed method reduces the major variations in measurements caused by incomplete FOV, compared to the baseline method. In the “Reference vs. proposed method” plots, measurements are tightly clustered near the middle-dashed line, demonstrating consistent and coherent agreement with the reference. Conversely, the “Reference vs. baseline” plots reveal a wider distribution of measurements along the y-axis, indicating considerable errors and variability. By ensuring consistent measurements of AD-associated bundles, the disclosed approach decreases the uncertainty in AD studies potentially compromised by incomplete FOV.

TABLE 2
Average Dice score for 72 tracts produced from the imputed images. The
improvement of the disclosed model over the baseline model is statistically
significant (all p-values is smaller than 0.01) from paired t-test
conducted for all-bundles and bundles associated with AD.

| | WRAP | | NACC | |
|---|---|---|---|---|
| | AD-associated | | AD-associated | |
| | bundles | All bundles | bundles | All bundles |
| baseline | 0.838 ± 0.049 | 0.844 ± 0.052 | 0.824 ± 0.066 | 0.839 ± 0.060 |
| disclosed method | 0.858 ± 0.039 | 0.865 ± 0.038 | 0.839 ± 0.054 | 0.854 ± 0.046 |
| p-value | * 0.0061 | * 2.0E−13 | * 0.0039 | * 2.7E−8 |

The disclosed method achieved completer and more accurate tractography compared with the baseline model, especially in regions close to the top edges of the brain (FIG. 6). The streamlines computed by the baseline model are either broken or stop propagating toward the top of the brain. However, the disclosed method addresses this issue by enabling the streamlines to propagate further and extend over a longer range, thus enhancing the visualization and analysis of neural pathways in whole brain areas. The quantitative results further support the improved imputation achieved by the disclosed method, demonstrated by consistent enhancements in average bundle segmentation across all bundles, including those associated with Alzheimer's Disease (Table 2). All improvements are statistically significant, with p-values less than 0.01, confirming the robustness of the disclosed method in imputing various neural pathways. Additionally, a detailed comparison of Dice scores achieved by the baseline and the disclosed models was conducted for each individual tract. Notably, the disclosed model consistently achieves improvement for almost all tracts, as evidenced by the ellipses of every tract predominantly positioned above the black dashed line, which denotes equal performance between baseline and the disclosed method (FIG. 7). The improvements are statistically significant for most tracts (p<0.01, highlighted by red centers) on both the WRAP and NACC datasets. The corresponding reference for each tract is presented in the right figure, where the color coding matches that of the ellipses, aiming for a clear visual correlation and aids in the rapid identification of each tract's accuracy metrics and its position in the brain. Finally, Bland-Altman plots for examining the shape measurements of the 12 AD associated bundles are presented in FIGS. 8A-8D. The disclosed approach shows significantly more consistent agreement with the reference measurements than the measurements obtained from images imputed by the baseline method, thereby reducing the uncertainty in analyzing bundles associated with Alzheimer's Disease.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A system for extending the field of view (FOV) of diffusion magnetic resonance imaging (dMRI) images, the system comprising:
non-transitory computer readable storage media that stores:
diffusion-weighted imaging (DWI) images of a patient having an incomplete field of view, the DWI images forming a plurality of volumes, each volume partitioned into a plurality of DWI image slices having an incomplete field of view; and
multi-modal data indicative of the shape of the brain of the patient;
a content module trained to extract diffusion features from each DWI image slice;
a shape module trained to extract shape information from the extracted diffusion features and the multi-modal data and output encoded features indicative of the extracted diffusion features and shape information; and
a spatial broadcast decoder trained to generate a synthesized DWI image slice having a complete field of view based on the encoded features extracted from each DWI image slice and the multi-modal data.

2. The system of claim 1, wherein the system generates the synthesized DWI images having the complete field of view by partitioning the each volume into DWI image slices having an incomplete field of view and, for each DWI image slice having an incomplete field of view, generating a synthesized DWI image slice having a complete field of view.

3. The system of claim 1, wherein the diffusion features are extracted from each DWI image slice and a predetermined number of adjacent DWI image slices.

4. The system of claim 1, wherein the multi-modal data comprises a T1 weighted (T1w) image of the patient having a complete FOV and a diffusion tensor image (DTI) orientation map.

5. The system of claim 1, wherein:
each volume is captured while applying distinct diffusion-encoding magnetic gradient pulses; and
for each of a plurality of volumes, the multi-modal data comprises a b-value indicative of the observed signal attenuation caused by the diffusion-encoding magnetic gradient pulses applied while capturing the volume and a b-vector map indicative of the direction of the diffusion-encoding magnetic gradient pulses applied while capturing the volume.

6. The system of claim 5, wherein:
the content module trained to extract diffusion features from each DWI image slice comprises a variational autoencoder (VAE) encoder that extracts an n-dimensional latent z-vector indicative of the signal intensity and texture properties of the entire DWI slice having the incomplete field of view.

7. The system of claim 6, wherein:
each synthesized DWI image slice is of dimension x×y; and
the content module broadcasts the extracted diffusion features to each pixel of the synthesized DWI image by generating a z-map having n channels, each of the n channels formed by tiling one of the n dimensions of the n-dimensional latent z-vector to form an array of dimension x×y.

8. The system of claim 7, wherein the shape module comprises a U-Net encoder and the spatial broadcast decoder comprises U-Net decoder with skip connections from the U-Net encoder.

9. The system of claim 8, wherein the U-Net encoder and the U-Net decoder generate each synthesized DWI image slice for each volume based on the n-channel z-map concatenated with the multi-modal data for the volume.

10. The system of claim 9, wherein:
the VAE encoder is trained on reference data to learn an inference model for extracting the n-dimensional latent z-vector; and
the U-Net encoder and the U-Net decoder are trained on the reference data to learn a generative model for generating the synthesized DWI image slices.

11. A method of extending the field of view (FOV) of diffusion-weighted imaging (DWI) images of a patient brain having an incomplete field of view, the DWI images forming a plurality of volumes, each volume partitioned into a plurality of DWI image slices having an incomplete field of view, the method comprising:
extracting diffusion features from each DWI image slice;
extracting shape information from the extracted diffusion features and multi-modal data of the patient to form encoded features indicative of the extracted diffusion features and shape information; and
generating a synthesized DWI image slice having a complete field of view based on the encoded features extracted from each DWI image slice and the multi-modal data.

12. The method of claim 1, further comprising:
partitioning each volume into DWI image slices having an incomplete field of view; and
generating a synthesized DWI image slice having a complete field of view for each DWI image slice having an incomplete field of view.

13. The method of claim 11, wherein extracting diffusion features from each DWI image slice comprises extracting diffusion features are extracted from each DWI image slice and a predetermined number of adjacent DWI image slices.

14. The method of claim 11, wherein the multi-modal data comprises a T1 weighted (T1w) image of the patient having a complete FOV and a diffusion tensor image (DTI) orientation map.

15. The method of claim 11, wherein:
each volume is captured while applying distinct diffusion-encoding magnetic gradient pulses; and
for each of a plurality of volumes, the multi-modal data comprises a b-value indicative of the observed signal attenuation caused by the diffusion-encoding magnetic gradient pulses applied while capturing the volume and a b-vector map indicative of the direction of the diffusion-encoding magnetic gradient pulses applied while capturing the volume.

16. The method of claim 15, wherein:
the extracted diffusion features extracted from each DWI image slice having the incomplete field of view are an n-dimensional latent z-vector indicative of the signal intensity and texture properties of the entire DWI slice extracted by a variational autoencoder (VAE) encoder.

17. The method of claim 6, wherein:
each synthesized DWI image slice is of dimension x×y; and
the method further comprises broadcasting the extracted diffusion features to each pixel of the synthesized DWI image by generating a z-map having n channels, each of the n channels formed by tiling one of the n dimensions of the n-dimensional latent z-vector to form an array of dimension x×y.

18. The method of claim 17, wherein the shape information is extracted by a U-Net encoder and the synthesized DWI image slice is generated by a U-Net decoder with skip connections from the U-Net encoder.

19. The method of claim 18, wherein the U-Net encoder and the U-Net decoder generate each synthesized DWI image slice for each volume based on the n-channel z-map concatenated with the multi-modal data for the volume.

20. The method of claim 19, wherein:
the VAE encoder is trained on reference data to lean an inference model for extracting the n-dimensional latent z-vector; and
the U-Net encoder and the U-Net decoder are trained on the reference data to learn a generative model for generating the synthesized DWI image slices.
